(19) United States
(12) Patent Application Publication
(10) Pub. No.: US 2024/0417390 A1
(43) Pub. Date: Dec. 19, 2024

(54) HYDROGEN SULFATE SALT OF A BCL-2 INHIBITOR, RELATED CRYSTALLINE FORM, METHOD FOR PREPARING THE SAME AND PHARMACEUTICAL COMPOSITIONS CONTAINING THE SAME

(71) Applicants: LES LABORATOIRES SERVIER, Suresnes Cedex (FR); VERNALIS (R&D) LTD, Cambridge (GB)

(72) Inventors: Michael LYNCH, Saint Jean De La Ruelle (FR); Frédéric VILLARD, Orléans (FR); Patrick MOUCHET, Ectot-L'auber (FR); Pascal TAULELLE, Le Havre (FR); Ludovic MASSON, Saint-Antoine-La-Foret (FR)

(21) Appl. No.: 18/816,347

(22) Filed: Aug. 27, 2024

Related U.S. Application Data
(63) Continuation of application No. 17/286,936, filed on Apr. 20, 2021, now abandoned.

(30) Foreign Application Priority Data
Oct. 31, 2018 (EP) 18306430.2

(51) Int. Cl. C07D 401/14

(52) U.S. Cl. CPC C07D 401/14; C07B 2200/13

(57) ABSTRACT

A salt and related crystalline forms of Compound A:

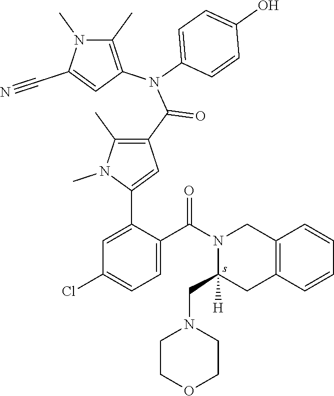 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]

wherein the salt is the hydrogen sulfate salt,

characterised by its X-ray powder diffraction diagram, method for preparing the same via crystallization, and pharmaceutical compositions containing it.

### FIELD OF THE INVENTION

The invention relates to a novel salt of 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide, referred to herein as ‘Compound A’, or polymorphs or solvates thereof, methods for preparing the same as well as pharmaceutical compositions thereof. In particular, the invention relates to the hydrogen sulfate salt of Compound A, referred to herein as ‘Compound A, H₂SO₄’, and the crystalline form I thereof. The present invention further discloses a process for preparing said crystalline form and pharmaceutical compositions comprising said crystalline form. The invention also relates to the use of such compositions for the treatment of cancer, diseases of the immune system and auto-immune diseases. Last, an anhydrous crystalline form of Compound A, H₂SO₄ is disclosed.

### BACKGROUND OF THE INVENTION

The chemical structure of Compound A is:

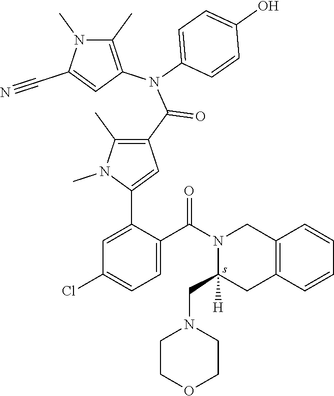 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]

Its preparation, its use as a Bcl-2 inhibitor for the treatment of cancer and pharmaceutical formulations thereof are described in WO 2015/011400 (Example 386), the content of which is incorporated by reference. The preparation of Compound A in the form of a hydrochloride salt (‘Compound A.HCl’) is specifically disclosed in this document. It is obtained as a lyophilisate.

Although Compound A is a very promising drug, it is a difficult compound to formulate. In particular, it is slightly soluble in water (<0.01 mg/ml for the free base). As a chemical substance can exhibit different physical properties being in one or another salt form or crystalline form thereof, this polymorphism of the drug molecule can affect the shelf life, solubility, formulation properties, processing properties, and the action of a drug. In addition, different polymorphs can have different rates of uptake in the body, leading to lower or higher biological activity than desired. In extreme cases, an undesired polymorph can even show toxicity. Understanding and controlling polymorphism, then, gives a decided advantage in bringing new drugs to the marketplace, which may be more active, more stable, or more cheaply manufactured. However, even though polymorphism has been a subject for intensive investigations, understanding and controlling this phenomenon represents a substantial scientific challenge. It is hard to predict whether a given molecule will crystallize in one or several crystal forms, and to find conditions leading to such crystallization.

From the industrial point of view, it is imperative to be able to synthesise the compound with excellent purity, and in particular in a highly reproducible form, having valuable characteristics of dissolution, filtration, drying, ease of formulation and stability enabling the prolonged storage thereof without particular requirements for temperature, light, humidity or oxygen levels.

The present invention also describes a process for obtaining Compound A, H₂SO₄ in a well-defined, perfectly reproducible crystalline form (Form I) having very good stability that is compatible with the industrial constraints of preparation, especially filtration, and storage of pharmaceutical compositions.

### BRIEF DESCRIPTION OF THE FIGURES

FIG. 1 shows the X-ray powder diffraction pattern (XPRD) of the crystalline form I of Compound A, H₂SO₄.

FIG. 2 shows the X-ray powder diffraction pattern (XPRD) of the anhydrous crystalline form of Compound A, hydrogen sulfate salt.

FIG. 3 shows the X-ray powder diffraction pattern (XPRD) of the crystalline form I of Compound A, hydrochloride salt

FIG. 4 shows the DSC and TGA profiles of the crystalline form I of Compound A, hydrogen sulfate salt

FIG. 5 shows the DSC and TGA profiles of the crystalline form I of Compound A, hydrochloride salt

FIG. 6 shows the solid-state ¹³C NMR spectrum of the crystalline form I of Compound A, H₂SO₄.

### DETAILED DESCRIPTION OF THE INVENTION

As used herein, the term ‘comprising’ means ‘including’, and is not intended to exclude the presence of any additional component, unless the context suggests otherwise, for example when the components together sum to 100%.

The term “alcohols” means C₁-C₆ alcohols such as methanol, ethanol, n-propanol, isopropanol, n-butanol, isobutanol, pentanol, 2-pentanol, 3-pentanol, isopentanol, hexanol.

‘Cancer’ means a class of disease in which a group of cells display uncontrolled growth. Cancer types include haematological cancers (lymphoma and leukemia) and solid tumors including carcinoma, sarcoma, or blastoma. ‘Cancer’ includes cancer of the bladder, brain, breast and uterus, chronic lymphoid leukaemias, colorectal cancer, cancers of the œsophagus and liver, lymphoblastic leukaemias, acute myeloid leukaemia, lymphomas, for example non-Hodgkin's B-cell lymphoma and diffuse large B-cell lymphoma, melanomas, malignant haemopathies, for example myelodysplastic syndrome, myelomas, for example multiple myeloma, ovarian cancer, non-small-cell lung cancer, prostate cancer, pancreatic cancer and small-cell lung cancer.

‘free molecule’ and ‘free base’ are used interchangeably herein and refer to Compound A when not in salt form.

### Embodiments of the invention

Described below are a number of embodiments of the invention.

E1. The hydrogen sulfate salt of 5-(5-chloro-2-{[(3S)-3-(morpholin-4-ylmethyl)-3,4-dihydroisoquinolin-2(1H)-yl] carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound A, H₂SO₄).

E2. A crystalline form I of the hydrogen sulfate salt of 5-(5-chloro-2-{[(3S)-3-(morpholin-4-ylmethyl)-3,4-dihydroisoquinolin-2(1H)-yl] carbonyl}phenyl)- N-(5-cyano-1,2-dimethyl-1H-pyrrol-3-yl)-N-(4-hydroxyphenyl)-1,2-dimethyl-1H-pyrrole-3-carboxamide (Compound A, H₂SO₄) according to E1, wherein the crystalline form has an X-ray powder diffraction diagram showing the following diffraction lines (Bragg's angle 2 theta, expressed in degrees ±0.2°): 5.55; 6.62 and 7.39.

E3. A crystalline form I of the hydrogen sulfate salt of 5-(5-chloro-2-{[(3S)-3-(morpholin-4-ylmethyl)-3,4-dihydroisoquinolin-2(1H)-yl]carbonyl}phenyl)-N- (5-cyano-1,2-dimethyl-1H-pyrrol-3-yl)-N-(4-hydroxyphenyl)-1,2-dimethyl-1H-pyrrole-3-carboxamide (Compound A, H₂SO₄) according to E1, wherein the crystalline form has an X-ray powder diffraction diagram showing at least 4, 5, 6, 7, 8, 9, 10, 11, 12, 13, 14 or all of the following diffraction lines (Bragg's angle 2 theta, expressed in degrees ±0.2°): 5.55; 5.62; 6.62; 7.39; 10.17; 11.49; 11.83; 16.01; 16.54; 17.04; 18.98; 19.18; 21.90; 22.28; 24.89.

E4. The crystalline form I of the hydrogen sulfate salt of Compound A according to E3, characterized in that it has an X-ray powder diffraction diagram having the following diffraction lines (Bragg's angle 2 theta, expressed in degrees ±0.2°): 5.55; 5.62; 6.62; 7.39; 10.17; 11.49; 11.83; 16.01; 16.54; 17.04; 18.98; 19.18; 21.90; 22.28; 24.89.

E5. The crystalline form I of the hydrogen sulfate salt of Compound A according to E4, characterized in that it has the following X-ray powder diffraction diagram, measured using a PANalytical X'Pert Pro MPD diffractometer with an X'Celerator detector and expressed in terms of line position (Bragg's angle 2 theta, expressed in degrees) +0.2° and interplanar distances d (expressed in Å):

| | Angle | Interplanar |
|---|---|---|
| Line | 2-theta | distance |
| no. | (degrees) | (Å) |
| 1 | 5.55 | 15.93 |
| 2 | 5.62 | 15.73 |
| 3 | 6.62 | 13.36 |
| 4 | 7.39 | 11.95 |
| 5 | 10.17 | 8.70 |
| 6 | 11.49 | 7.70 |
| 7 | 11.83 | 7.48 |
| 8 | 16.01 | 5.53 |
| 9 | 16.54 | 5.36 |
| 10 | 17.04 | 5.20 |
| 11 | 18.98 | 4.67 |
| 12 | 19.18 | 4.63 |
| 13 | 21.90 | 4.06 |
| 14 | 22.28 | 3.99 |
| 15 | 24.89 | 3.58 |

E6. The crystalline form I of the hydrogen sulfate salt of Compound A according to any one of E1 to E5, characterised in that it has a solid-state ¹³C CP/MAS NMR spectrum having the following peaks (expressed in ppm±0.2 ppm): 173.31 ppm, 155.32 ppm, 140.46 ppm, 139.19 ppm, 137.42 ppm, 134.68 ppm, 131.65 ppm, 131.14 ppm, 129.37 ppm, 126.32 ppm, 118.77 ppm, 117.36 ppm, 116.54 ppm, 113.61 ppm, 112.69 ppm, 110.74 ppm, 102.33 ppm, 101.45 ppm, 63.06 ppm, 57.19 ppm, 54.87 ppm, 52.06 ppm, 44.71 ppm, 43.94 ppm, 34.42 ppm, 32.89 ppm, 31.28 ppm, 30.66 ppm, 14.40 ppm, 13.34 ppm, 12.49 ppm and 10.50 ppm.

E7. Pharmaceutical composition comprising as active ingredient the hydrogen sulfate salt of Compound A according to E1 in association with one or more pharmaceutically acceptable excipients.

E8. Pharmaceutical composition comprising as active ingredient the crystalline form I of the hydrogen sulfate salt of Compound A according to any one of E2 to E6 in association with one or more pharmaceutically acceptable excipients.

E9. Pharmaceutical composition according to E7 or E8 for use in the treatment of cancers, auto-immune diseases and diseases of the immune system.

E10. Pharmaceutical composition according to E9, wherein the cancer is selected from the bladder, brain, breast and uterus cancers, chronic lymphoid leukaemias, colorectal cancer, cancers of the œesophagus and liver, lymphoblastic leukaemias, acute myeloid leukaemia, lymphomas, for example non-Hodgkin's B-cell lymphoma and diffuse large B-cell lymphoma, melanomas, malignant haemopathies, for example myelodysplastic syndrome, myelomas, for example multiple myeloma, ovarian cancer, non-small-cell lung cancer, prostate cancer, pancreatic cancer and small-cell lung cancer.

E11. The hydrogen sulfate salt of Compound A according to E1 for use as a medicament.

E12. The hydrogen sulfate salt of Compound A according to E1 for use in the treatment of cancers, auto-immune diseases and diseases of the immune system.

E13. The hydrogen sulfate salt of Compound A according to E12 wherein the cancer is selected from the bladder, brain, breast and uterus cancers, chronic lymphoid leukaemias, colorectal cancer, cancers of the œsophagus and liver, lymphoblastic leukaemias, acute myeloid leukaemia, lymphomas, for example non-Hodgkin's B-cell lymphoma and diffuse large B-cell lymphoma, melanomas, malignant haemopathies, for example myelodysplastic syndrome, myelomas, for example multiple myeloma, ovarian cancer, non-small-cell lung cancer, prostate cancer, pancreatic cancer and small-cell lung cancer.

E14. The crystalline form I of the hydrogen sulfate salt of Compound A according to any one of E2 to E6 for use as a medicament.

E15. The crystalline form I of the hydrogen sulfate salt of Compound A according to any one of E2 to E6 for use in the treatment of cancers, auto-immune diseases and diseases of the immune system.

E16. The crystalline form I of the hydrogen sulfate salt of Compound A according to E15 wherein the cancer is selected from the bladder, brain, breast and uterus cancers, chronic lymphoid leukaemias, colorectal cancer, cancers of the œesophagus and liver, lymphoblastic leukaemias, acute myeloid leukaemia, lymphomas, for example non-Hodgkin's B-cell lymphoma and diffuse large B-cell lymphoma, melanomas, malignant haemopathies, for example myelodysplastic syndrome, myelomas, for example multiple myeloma, ovarian cancer, non-small-cell lung cancer, prostate cancer, pancreatic cancer and small-cell lung cancer.

E17. Process for the preparation of the crystalline form I of the hydrogen sulfate salt of Compound A according to any one of E2 to E6, wherein the hydrogen sulfate salt of Compound A is crystallised in a polar medium.

E18. Process for the preparation of the crystalline form I of the hydrogen sulfate salt of Compound A according to E17, wherein the polar medium is composed of one or more solvents selected from water and alcohols.

E19. Process for the preparation of the crystalline form I of the hydrogen sulfate salt of Compound A according to E18, wherein the alcohol is ethanol.

E20. Process for the preparation of the crystalline form I of the hydrogen sulfate salt of Compound A according to E18, wherein the polar medium is an ethanol/water mixture.

E21. Process for the preparation of the crystalline form I of the hydrogen sulfate salt of Compound A according to any one of E17 to E20, in which process the crystallisation is seeded using a very small amount of the crystalline form I of the hydrogen sulfate salt of Compound A.

E22. Anhydrous crystalline form of the hydrogen sulfate salt of 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound A, H₂SO₄) according to E1, wherein the crystalline form has an X-ray powder diffraction diagram showing at least 4, 5, 6, 7, 8, 9, 10, 11, 12, 13, 14, 15 or all of the following diffraction lines (Bragg's angle 2 theta, expressed in degrees ±0.2°): 5.19; 5.64; 6.74; 7.14; 8.04; 8.33; 9.17; 9.40; 10.68; 11.03; 11.35; 12.18; 12.59; 13.64; 14.78; 15.09.

E23. The anhydrous crystalline form according to E22, characterized in that it has the following X-ray powder diffraction diagram, measured using a PANalytical X'Pert Pro MPD diffractometer with an X'Celerator detector and expressed in terms of line position (Bragg's angle 2 theta, expressed in degrees ±0.2°) and interplanar distances d (expressed in Å):

| | Angle | Interplanar |
|---|---|---|
| Line | 2-theta | distance |
| no. | (degrees) | (Å) |
| 1 | 5.19 | 17.03 |
| 2 | 5.64 | 15.66 |
| 3 | 6.74 | 13.12 |
| 4 | 7.14 | 12.39 |
| 5 | 8.04 | 10.99 |
| 6 | 8.33 | 10.61 |
| 7 | 9.17 | 9.64 |
| 8 | 9.40 | 9.41 |
| 9 | 10.68 | 8.29 |
| 10 | 11.03 | 8.02 |
| 11 | 11.35 | 7.79 |
| 12 | 12.18 | 7.26 |
| 13 | 12.59 | 7.03 |
| 14 | 13.64 | 6.49 |
| 15 | 14.78 | 5.99 |
| 16 | 15.09 | 5.87 |

Obtaining the crystalline form I of the hydrogen sulfate salt of Compound A has the advantage of having good characteristics of stability. More especially, only one crystalline form was observed in the range of solvents and temperatures used for the screening, showing a limited polymorphism of the hydrogen sulfate salt in the tested conditions. Furthermore, the crystalline form I of the hydrogen sulfate salt of Compound A thereby obtained is sufficiently stable to allow its storage for an extended period without particular conditions for temperature, light, humidity or oxygen levels.

The Examples herein below illustrate the invention but do not limit it in any way.

### Example 1: Process for Obtaining Crystalline Form I of the Hydrogen Sulfate Salt of Compound A

25 g of Compound A (free base) was placed in 239.5 g of ethanol at ambient temperature. The mixture was then heated at 65° C. A solution of sulphuric acid in water (4.27 g of H₂SO₄ +59.87 g of water) was then added gradually. The mixture was stirred for 1 h before being cooled to 10° C. When the crystallisation was complete, the suspension was filtered, washed with an ethanol/water mixture à 10° C., filtered and dried under reduced pressure. After drying, crystalline form I of the hydrogen sulfate salt of Compound A was obtained in a yield of about 70% and with a purity greater than 99.8%. The solid was characterised by the X-ray powder as set out in Example 3.

In the crystallisation process according to the invention, Compound A (free base) is obtained by any process which may be used.

### Example 2: Process for Obtaining Crystalline Form I of the Hydrogen Sulfate Salt of Compound A (Seeding)

25 g of Compound A (free base) was placed in 239.5 g of ethanol at ambient temperature. The mixture was then heated at 65° C. A solution of sulphuric acid in water (4.27 g of H₂SO₄ +59.87 g of water) was then added gradually. The mixture was stirred for 30 minutes. The mixture was then cooled slightly before being seeded with the crystalline form I of the hydrogen sulfate salt of Compound A (2% by weight of starting material). The mixture was stirred for 30 minutes before being cooled to 10° C. When the crystallisation was complete, the suspension was filtered, washed with an ethanol/water mixture à 10° C., filtered and dried under reduced pressure. After drying, crystalline form I of the hydrogen sulfate salt of Compound A was obtained in a yield of about 70% and with a purity greater than 99.8%. The solid was characterised by the X-ray powder as set out in Example 3.

In the crystallisation process according to the invention, Compound A (free base) is obtained by any process which may be used.

### Example 3: Crystalline Form I of the Hydrogen Sulfate Salt of Compound A (X-Ray Powder Diffraction Diagram)

Recording of the data was carried out in the transmission mode using a PANalytical X'Pert Pro MPD diffractometer with an X'Celerator detector under the following conditions:
Voltage 45 kV, current 40 mA,
Mounting: theta/theta,
Anode: copper,
K alpha-1 wavelength: 1.54060 Å,
K alpha-2 wavelength: 1.54443 Å,
K alpha-2/K alpha-1 ratio: 0.5,
Measurement mode: continuous from 3° to 55° (Bragg's angle 2 theta) in increments of 0.017°,
Measurement time per step: 35.5301 s.

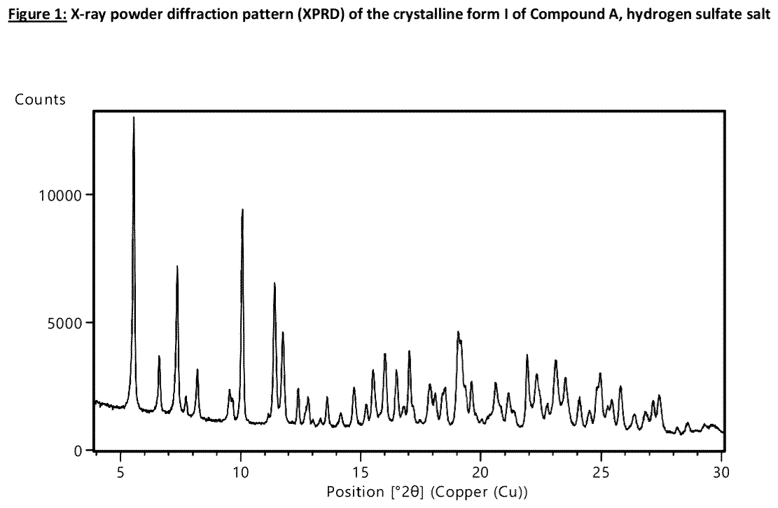

The X-ray powder diffraction diagram of the form I of the hydrogen sulfate salt of Compound A obtained according to the process of Example 1 or 2 is expressed in terms of line position (Bragg's angle 2 theta, expressed in degrees ±0.2°) and interplanar distances (expressed in Å) (FIG. 1). The significant lines have been collated in the following table:

| | Angle | Interplanar |
|---|---|---|
| Line | 2-theta | distance |
| no. | (degrees) | (Å) |
| 1 | 5.55 | 15.93 |
| 2 | 5.62 | 15.73 |
| 3 | 6.62 | 13.36 |
| 4 | 7.39 | 11.95 |
| 5 | 10.17 | 8.70 |
| 6 | 11.49 | 7.70 |
| 7 | 11.83 | 7.48 |
| 8 | 16.01 | 5.53 |
| 9 | 16.54 | 5.36 |
| 10 | 17.04 | 5.20 |
| 11 | 18.98 | 4.67 |
| 12 | 19.18 | 4.63 |
| 13 | 21.90 | 4.06 |
| 14 | 22.28 | 3.99 |
| 15 | 24.89 | 3.58 |

### Example 4: Stability Studies

For all storage conditions and storage periods, 20 mg of crystalline form of the salt of Compound A were introduced in a 30-mL vial for post-storage HPLC analysis.

The drug substance content was determined by LC (% m/m).

| | | Hydrogen sulfate salt, |
|---|---|---|
| Temperature | Packaging | crystalline form I |
| T₀ | | >99.9 |
| 25° C./60% RH | Double polyethylene | 100.7 after 3 months |
| | bag placed | of storage |
| | in a plastic drum | |
| 30° C./65% RH | Double polyethylene | 100.6 after 3 months |
| | bag placed | of storage |
| | in a plastic drum | |
| 40° C./75% RH | Double polyethylene | 100.0 after 3 months |
| | bag placed | of storage |
| | in a plastic drum | |
| 50° C./75% RH | Open glass bottle | 101.0 after 6 weeks |
| | | of storage |

The appearance of the powder (white) and chemical stability remains unchanged under all conditions tested: over 3 months at 25° C./60% RH, 30° C./65% RH, 40° C./75% RH, and for 6 weeks at 50° C./75% RH.

Furthermore, the X-ray diffraction results show that the form does not change after analysis at To and after 6 weeks storage in open glass bottles at 25° C./90% RH. In conclusion, the drug substance can be considered physically and chemically stable over the periods tested.

### Example 5: Process for Obtaining the Anhydrous Crystalline Form of the Hydrogen Sulfate Salt of Compound A (Seeding)

5.83 kg of Compound A (free base) was placed in 55.85 kg of ethanol at ambient temperature. The mixture was then heated at 65° C. A solution of sulphuric acid in water (1 kg of H₂SO₄+13.96 kg of water) was then added gradually. The mixture was stirred for 30 minutes. The mixture was then cooled slightly before being seeded with the crystalline form I of the hydrogen sulfate salt of Compound A (2% by weight of starting material). The mixture was stirred for 30 minutes before being cooled to 10° C. When the crystallisation was complete, the suspension was filtered, washed with an ethanol/water mixture à 10° C., filtered and dried under reduced pressure. Then, the dried product is stored under an inert atmosphere (nitrogen). The anhydrous crystalline form of the hydrogen sulfate salt of Compound A was obtained in a yield of about 78±5% and with a purity greater than 99.9% and a water content of about 0.43%. The solid was characterised by the X-ray powder as set out in Example 6.

In the crystallisation process according to the invention, Compound A (free base) is obtained by any process which may be used.

Example 6: Anhydrous Crystalline Form of the Hydrogen Sulfate Salt of Compound A (X-Ray Powder Diffraction Diagram)

Recording of the data was carried out in the following conditions:

Approximately 30-50 mg of the sample to be analysed is placed between two polymeric films (Kapton®) fixed in a sample holder disc. The X-ray diffraction pattern of the test sample is recorded from 3∪ 2theta to at least 40° 2-theta in 15 min using an X-ray diffractometer operating in the transmission mode with CuKα radiation (λ=1.5418 Å) at 40 kV and 30 mA and with a step size ranging from 0.01 to 0.02° 2-theta. These settings may vary according to the diffractometer used.

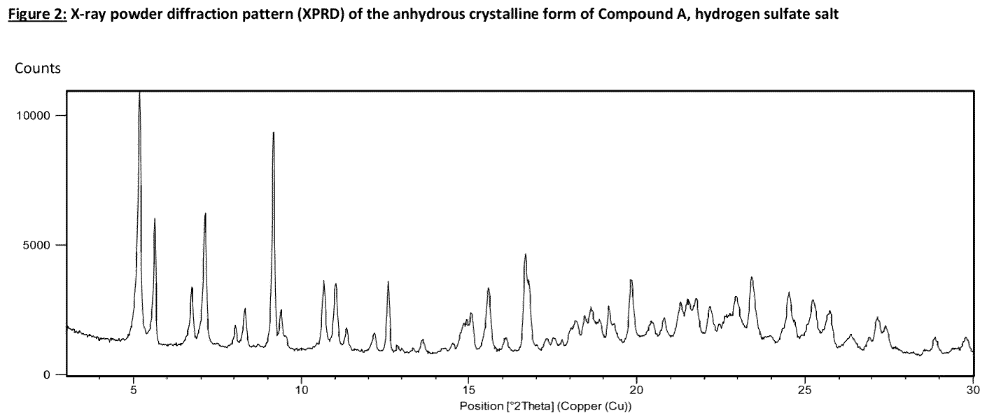

The X-ray powder diffraction diagram of the anhydrous form of the hydrogen sulfate salt of Compound A obtained according to the process of Example 5 is expressed in terms of line position (Bragg's angle 2 theta, expressed in degrees ±0.2°) and interplanar distances (expressed in Å) (FIG. 2). The significant lines have been collated in the following table:

| | Angle | Interplanar |
|---|---|---|
| Line | 2-theta | distance |
| no. | (degrees) | (Å) |
| 1 | 5.19 | 17.03 |
| 2 | 5.64 | 15.66 |
| 3 | 6.74 | 13.12 |
| 4 | 7.14 | 12.39 |
| 5 | 8.04 | 10.99 |
| 6 | 8.33 | 10.61 |
| 7 | 9.17 | 9.64 |
| 8 | 9.40 | 9.41 |
| 9 | 10.68 | 8.29 |
| 10 | 11.03 | 8.02 |
| 11 | 11.35 | 7.79 |
| 12 | 12.18 | 7.26 |
| 13 | 12.59 | 7.03 |
| 14 | 13.64 | 6.49 |
| 15 | 14.78 | 5.99 |
| 16 | 15.09 | 5.87 |

### Example 7: Process for Obtaining Crystalline Form I of the Hydrogen Sulfate Salt of Compound A (Seeding, Batch Size of the Order of the Kilogram)

5.83 kg of Compound A (free base) was placed in 55.85 kg of ethanol at ambient temperature. The mixture was then heated at 65° C. A solution of sulphuric acid in water (1 kg of H₂SO₄+13.96 kg of water) was then added gradually. The mixture was stirred for 30 minutes. The mixture was then cooled slightly before being seeded with the crystalline form I of the hydrogen sulfate salt of Compound A (2% by weight of starting material). The mixture was stirred for 30 minutes before being cooled to 10° C. When the crystallisation was complete, the suspension was filtered, washed with an ethanol/water mixture at 10° C., filtered and dried under reduced pressure. The product was rehydrated thereafter at 40° C. under an atmosphere with 50% of relative humidity (RH). The resulting product was stored under an inert atmosphere (nitrogen). The crystalline form I of the hydrogen sulfate salt of Compound A was obtained in a yield of about 78±5% and with a purity greater than 99.9% and a water content of about 6.5±1%. The solid was characterised by the X-ray powder as set out in Example 3.

In the crystallisation process according to the invention, Compound A (free base) is obtained by any process which may be used.

### Example 8: Process for Obtaining the Crystalline Form I of the Hydrochloride Salt of Compound A and the X-Ray Powder Diffraction Diagram Characterising It

1510 mg of the amorphous hydrochloride salt of Compound A (Example 386 of WO 2015/011400) was converted into its crystalline ethanol solvate by slurrying in 15 ml of ethanol for 48 h hours. The residual solid was filtered, washed twice with 1 ml of ethanol and then suspended in 10 ml of water for 5 min. After a difficult filtration, the residual solid was dried overnight at 30°° C./10 mbar and analysed by X-ray diffraction (3-30° 2 theta/10 min).

The mode of preparation for the HCl salt is complicated by the fact that it initially results in an ethanol solvate which is replaced by H2O after resuspension in water to give the hydrated form. The resulting hydrated HCl salt formed fine needles which were quite difficult to filter.

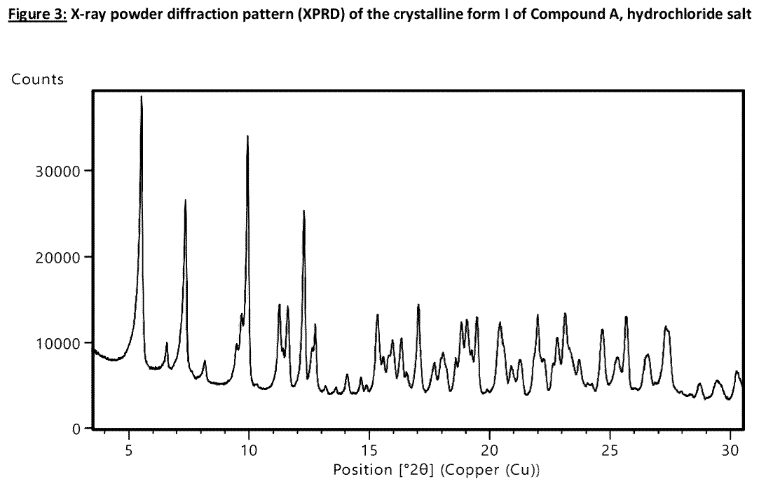

The X-ray powder diffraction diagram of the form I of the hydrochloride salt of Compound A obtained according to the process described previously is expressed in terms of line position (Bragg's angle 2 theta, expressed in degrees ±0.2°) and relative intensity (expressed in %) (FIG. 3). The significant lines have been collated in the following table:

| | Angle | Relative |
|---|---|---|
| Line | 2-theta | Intensity |
| no. | (degrees) | (%) |
| 1 | 5.53 | 100.00 |
| 2 | 7.37 | 65.92 |
| 3 | 9.96 | 92.98 |
| 4 | 11.26 | 31.90 |
| 5 | 11.62 | 30.11 |
| 6 | 12.29 | 67.53 |
| 7 | 12.76 | 25.47 |
| 8 | 15.34 | 29.27 |
| 9 | 17.04 | 32.91 |
| 10 | 18.82 | 25.98 |
| 11 | 19.07 | 27.10 |
| 12 | 19.48 | 27.89 |
| 13 | 20.41 | 25.05 |
| 14 | 21.99 | 28.88 |
| 15 | 23.14 | 29.52 |
| 16 | 24.69 | 23.99 |
| 17 | 25.66 | 29.09 |
| 18 | 27.28 | 25.49 |

### Example 9: DSC and TGA Profiles of the Crystalline Forms I of the Hydrochloride and Hydrogen Sulfate Salts of Compound A

### H₂SO₄ Salt

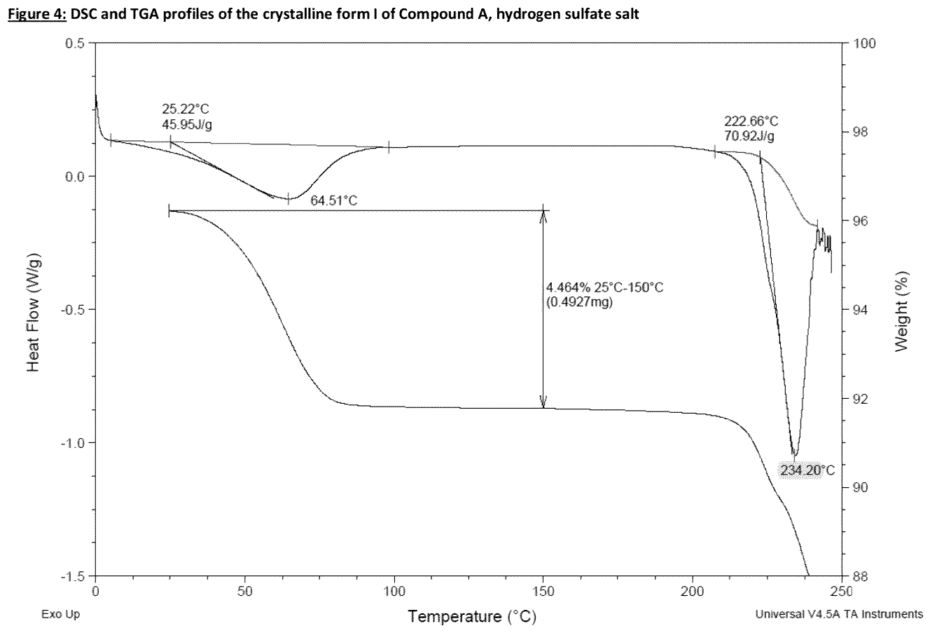

The Differential Scanning calorimetry (DSC) profile of a sample of the hydrogensulfate salt, form I weighing approximately 4 mg was recorded between 0°° C. and 250° C. at 10° C./min in pin-hole pierced aluminium pans under a positive flow of nitrogen on a TA Instruments Q1000 (or Q2000) Differential Scanning calorimeter (FIG. 4).

The Thermal Gravimetric Analysis (TGA) profile of a sample of the hydrogensulfate salt, form I weighing approximately 10 mg was recorded between 25° C. and 250° C. at 10° C./min in an open aluminium pan under a positive flow of nitrogen on a TA Instruments Q5000Thermogravimetric Analyser (FIG. 4).

### HCl Salt

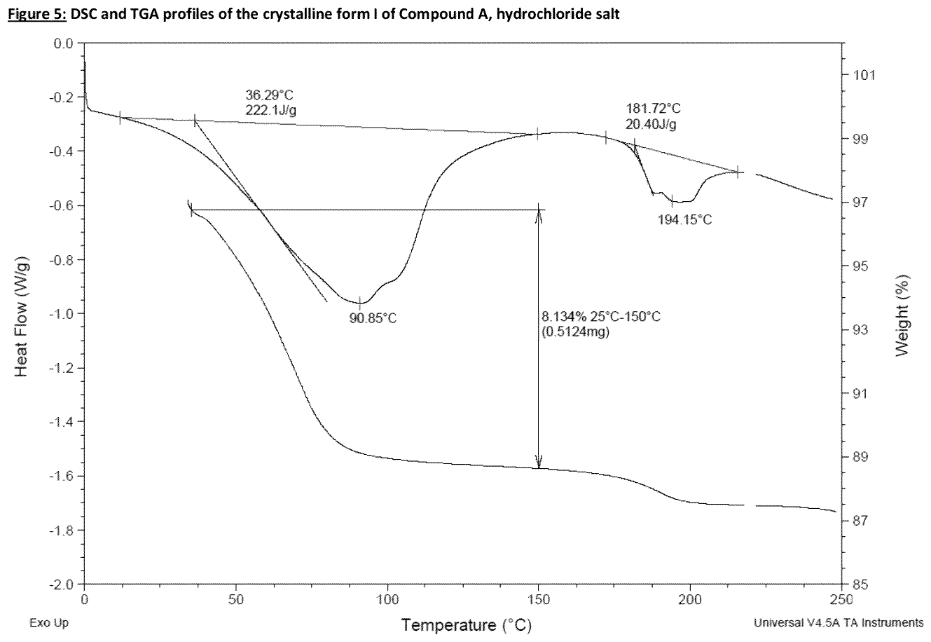

The DSC profile of a sample of the hydrochloride salt, form I weighing approximately 4 mg was recorded between 0° C. and 250° C. at 10° C./min in pin-hole pierced aluminium pans under a positive flow of nitrogen on a TA Instruments Q1000 (or Q2000) Differential Scanning calorimeter (FIG. 5).

The TGA profile of a sample of the hydrochloride salt, form I weighing approximately 6 mg was recorded between 25° C. and 250° C. at 10° C./min in an open aluminium pan under a positive flow of nitrogen on a TA Instruments Q5000 Thermogravimetric Analyser (FIG. 5).

The DSC profile of the H₂SO₄ salt is less complicated compared to that of the HCl salt. Water loss is visible in the TGA profile of the H₂ SO₄ salt between 25 and 100° C. A melting/degradation endotherm is visible in the DSC profile towards 224° C. The melting temperature and enthalpy of the HCl salt is lower than that of the H₂SO₄ salt. This may suggest that the HCl has a lower degree of crystallinity following dehydration compared to the H₂SO₄ salt.

### Example 10: Crystalline Form I of Compound A, H₂SO₄ (Solid NMR Spectrum)

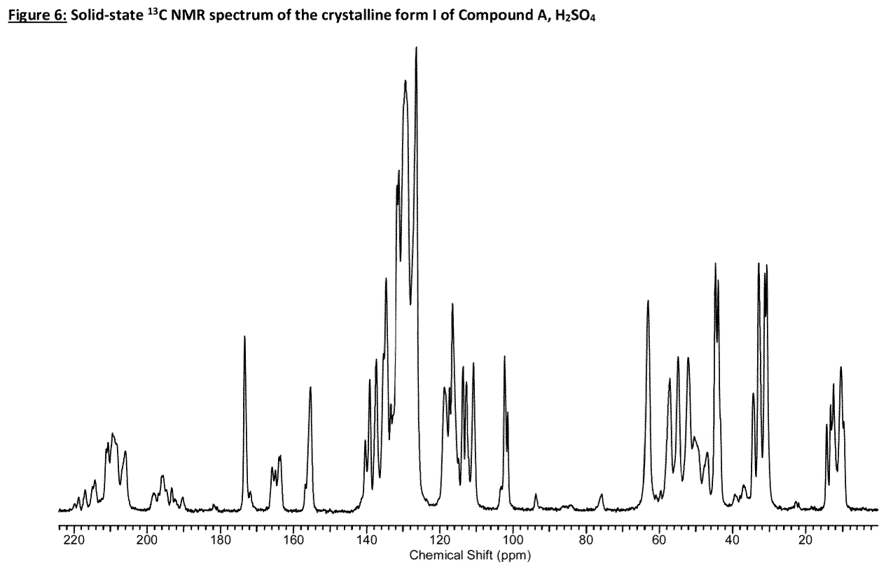

Crystalline form I of Compound A, H₂SO₄ was also characterized by solid-state Nuclear Magnetic Resonance spectroscopy (FIG. 6). Solid-state ¹³C NMR spectra of Compound A, H₂SO₄ were recorded at ambient temperature using a Bruker SB Avance III HD 500 spectrometer with a 4 mm CP/MAS SB VTN type probe under the following conditions:
Frequency: 125.76 MHZ,
Spectral width: 37 kHz,
Magic angle spinning rate: 10 kHz,
Pulse program: Cross Polarization with SPINAL64 decoupling
Recycle delay: 10 s,
Acquisition time: 46 ms,
Contact time: 4 ms,
Number of scans: 4096.

A 5 Hz line-broadening was applied prior to Fourier Transformation.

The spectrum thereby obtained was referenced relative to a sample of adamantane (the high frequency peak of adamantane was set to 38.5 ppm).

Crystalline form I of Compound A, H₂SO₄ can be defined by the presence of a set of peaks whose chemical shifts are given in the table below (expressed in ppm±0.2 ppm):

| No. | (ppm) |
|---|---|
| 1 | 173.31 |
| 2 | 155.32 |
| 3 | 140.46 |
| 4 | 139.19 |
| 5 | 137.42 |
| 6 | 134.68 |
| 7 | 131.65 |
| 8 | 131.14 |
| 9 | 129.37 |
| 10 | 126.32 |
| 11 | 118.77 |
| 12 | 117.36 |
| 13 | 116.54 |
| 14 | 113.61 |
| 15 | 112.69 |
| 16 | 110.74 |
| 17 | 102.33 |
| 18 | 101.45 |
| 19 | 63.06 |
| 20 | 57.19 |
| 21 | 54.87 |
| 22 | 52.06 |
| 23 | 44.71 |
| 24 | 43.94 |
| 25 | 34.42 |
| 26 | 32.89 |
| 27 | 31.28 |
| 28 | 30.66 |
| 29 | 14.40 |
| 30 | 13.34 |
| 31 | 12.49 |
| 32 | 10.50 |

1. A hydrogen sulfate salt of 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl- 1H-pyrrol-3-yl)-N-(4-hydroxyphenyl)-1,2-dimethyl-1H-pyrrole-3-carboxamide (Compound A, H₂SO₄).

2. A crystalline form I of the hydrogen sulfate salt of 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound A, H₂SO₄) according to claim 1, having an X-ray powder diffraction diagram which exhibits the following diffraction lines (Bragg's angle 2 theta, expressed in degrees) +0.2°: 5.55; 6.62 and 7.39.

3. A crystalline form I of the hydrogen sulfate salt of 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (Compound A, H2SO4) according to claim 1, having an X-ray powder diffraction diagram which exhibits at least 4, 5, 6, 7, 8, 9, 10, 11, 12, 13, 14 or all of the following diffraction lines (Bragg's angle 2 theta, expressed in degrees ±0.2°): 5.55; 5.62; 6.62; 7.39; 10.17; 11.49; 11.83; 16.01; 16.54; 17.04; 18.98; 19.18; 21.90; 22.28; 24.89.

4. The crystalline form I of the hydrogen sulfate salt of Compound A according to claim 3, having an X-ray powder diffraction diagram which exhibits the following diffraction lines (Bragg's angle 2 theta, expressed in degrees ±0.2°): 5.55; 5.62; 6.62; 7.39; 10.17; 11.49; 11.83; 16.01; 16.54; 17.04; 18.98; 19.18; 21.90; 22.28; 24.89.

5. The crystalline form I of the hydrogen sulfate salt of Compound A according to claim 4, having the following X-ray powder diffraction diagram, measured using a PANalytical X'Pert Pro MPD diffractometer with an X'Celerator detector and expressed in terms of line position (Bragg's angle 2 theta, expressed in degrees ±0.2°) and interplanar distances d (expressed in Å):

| | Angle | Interplanar |
|---|---|---|
| Line | 2-theta | distance |
| no. | (degrees) | (Å) |
| 1 | 5.55 | 15.93 |
| 2 | 5.62 | 15.73 |
| 3 | 6.62 | 13.36 |
| 4 | 7.39 | 11.95 |
| 5 | 10.17 | 8.70 |
| 6 | 11.49 | 7.70 |
| 7 | 11.83 | 7.48 |
| 8 | 16.01 | 5.53 |
| 9 | 16.54 | 5.36 |
| 10 | 17.04 | 5.20 |
| 11 | 18.98 | 4.67 |
| 12 | 19.18 | 4.63 |
| 13 | 21.90 | 4.06 |
| 14 | 22.28 | 3.99 |
| 15 | 24.89 | 3.58 |

6. The crystalline form I of the hydrogen sulfate salt of Compound A according to claim 2, having a solid-state ¹³C CP/MAS NMR spectrum which exhibits the following peaks (expressed in ppm±0.2 ppm): 173.31 ppm, 155.32 ppm, 140.46 ppm, 139.19 ppm, 137.42 ppm, 134.68 ppm, 131.65 ppm, 131.14 ppm, 129.37 ppm, 126.32 ppm, 118.77 ppm, 117.36 ppm, 116.54 ppm, 113.61 ppm, 112.69 ppm, 110.74 ppm, 102.33 ppm, 101.45 ppm, 63.06 ppm, 57.19 ppm, 54.87 ppm, 52.06 ppm, 44.71 ppm, 43.94 ppm, 34.42 ppm, 32.89 ppm, 31.28 ppm, 30.66 ppm, 14.40 ppm, 13.34 ppm, 12.49 ppm and 10.50 ppm.

7. A pharmaceutical composition comprising as active ingredient the hydrogen sulfate salt of Compound A according to claim 1 in combination with one or more pharmaceutically acceptable excipients.

8. A pharmaceutical composition comprising as active ingredient the crystalline form I of the hydrogen sulfate salt of Compound A according to claim 2 in combination with one or more pharmaceutically acceptable excipients.

9. A method of treating a condition selected from bladder cancer, brain cancer, breast cancer, uterus cancer, chronic lymphoid leukaemias, colorectal cancer, cancers of the esophagus and liver, lymphoblastic leukaemias, acute myeloid leukaemia, lymphomas, for example non-Hodgkin's B-cell lymphoma and diffuse large B-cell lymphoma, melanomas, malignant haemopathies, including myelodysplastic syndrome, myelomas, including multiple myeloma, ovarian cancer, non-small-cell lung cancer, prostate cancer, pancreatic cancer and small-cell lung cancer in a subject in need thereof, comprising administration of an effective amount of the hydrogen sulfate salt of Compound A according to claim 1, alone or in combination with one or more pharmaceutically acceptable excipients.

10. A method of treating a condition selected from bladder cancer, brain cancer, breast cancer, uterus cancer, chronic lymphoid leukaemias, colorectal cancer, cancers of the esophagus and liver, lymphoblastic leukaemias, acute myeloid leukaemia, lymphomas, for example non-Hodgkin's B-cell lymphoma and diffuse large B-cell lymphoma, melanomas, malignant haemopathies, including myelodysplastic syndrome, myelomas, including multiple myeloma, ovarian cancer, non-small-cell lung cancer, prostate cancer, pancreatic cancer and small-cell lung cancer in a subject in need thereof, comprising administration of an effective amount of the crystalline form I of the hydrogen sulfate salt of Compound A according to claim 2, alone or in combination with one or more pharmaceutically acceptable excipients.

11. A process for the preparation of the crystalline form I of the hydrogen sulfate salt of Compound A according to claim 2, wherein the hydrogen sulfate salt of Compound A is crystallised in a polar medium.

12. The process according to claim 11, wherein the polar medium is composed of one or more solvents selected from water and alcohols.

13. The process according to claim 12, wherein the alcohol is ethanol.

14. The process according to claim 12, wherein the polar medium is an ethanol/water mixture.

15. The process according to claim 11, in which process the crystallisation is seeded using a very small amount of the crystalline form I of the hydrogen sulfate salt of Compound A.

16. An anhydrous crystalline form of the hydrogen sulfate salt of 5-(5-chloro-2-{[(3S)-3-(morpholin-4-ylmethyl)-3,4-dihydroisoquinolin-2(1H)-yl]carbonyl}phenyl)- N-(5-cyano-1,2-dimethyl-1H-pyrrol-3-yl)-N-(4-hydroxyphenyl)-1,2-dimethyl-1H-pyrrole-3-carboxamide (Compound A, H2SO4) according to claim 1, having an X-ray powder diffraction diagram which exhibits at least 4, 5, 6, 7, 8, 9, 10, 11, 12, 13, 14, 15 or all of the following diffraction lines (Bragg's angle 2 theta, expressed in degrees ±0.2°): 5.19; 5.64; 6.74; 7.14; 8.04; 8.33; 9.17; 9.40; 10.68; 11.03; 11.35; 12.18; 12.59; 13.64; 14.78; 15.09.

17. The anhydrous crystalline form of the hydrogen sulfate salt of Compound A according to claim 16, having the following X-ray powder diffraction diagram, measured using a PANalytical X'Pert Pro MPD diffractometer with an X'Celerator detector and expressed in terms of line position (Bragg's angle 2 theta, expressed in degrees ±0.2°) and interplanar distances d (expressed in Å):

| | Angle | Interplanar |
|---|---|---|
| Line | 2-theta | distance |
| no. | (degrees) | (Å) |
| 1 | 5.19 | 17.03 |
| 2 | 5.64 | 15.66 |
| 3 | 6.74 | 13.12 |
| 4 | 7.14 | 12.39 |
| 5 | 8.04 | 10.99 |
| 6 | 8.33 | 10.61 |
| 7 | 9.17 | 9.64 |
| 8 | 9.40 | 9.41 |
| 9 | 10.68 | 8.29 |
| 10 | 11.03 | 8.02 |
| 11 | 11.35 | 7.79 |
| 12 | 12.18 | 7.26 |
| 13 | 12.59 | 7.03 |
| 14 | 13.64 | 6.49 |
| 15 | 14.78 | 5.99 |
| 16 | 15.09 | 5.87 |
